(19) United States
(12) Patent Application Publication
(10) Pub. No.: US 2024/0390668 A1
(43) Pub. Date: Nov. 28, 2024

(54) CURED RING SEAL

(71) Applicant: Cochlear Limited, Macquarie University, NSW (AU)

(72) Inventor: Jonathon KIRK, Sydney (AU)

(21) Appl. No.: 18/686,108

(22) PCT Filed: Aug. 12, 2022
(86) PCT No.: PCT/IB2022/057557

§ 371 Date: Feb. 23, 2024

Related U.S. Application Data
(60) Provisional application No. 63/239,580, filed on Sep. 1, 2021.

(51) Int. Cl. A61N 1/05; A61M 31/00; F16J 15/10

(52) U.S. Cl. CPC A61N 1/0541; A61M 31/00; F16J 15/102; A61M 2210/0662

(57) ABSTRACT

Presented herein are cured ring seals and related methods for at least temporarily sealing openings in fluidically-sealed body chambers. The cured ring seals include a body that is formed from a hydrophilic or hygroscopic polymer, such as polyvinyl alcohol (PVA), that is cured prior to implantation into the body of a recipient (e.g., pre-cured). The cured ring seals are substantially pliable to facilitate positioning around an outer surface of an implantable component and are configured to expand following implantation.

### BACKGROUND

### Field of the Invention

The present invention relates generally to cured ring seals for body chambers.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a cured ring seal is provided. The ring seal is configured to fluidically seal an opening of a body chamber of a human body, wherein the seal is shaped to overlap the outer edges of the opening and wherein the seal has an aperture through which an implantable component is inserted into the body chamber.

In another example, a method is provided. The method comprises: accessing an opening to a fluidically-sealed body chamber in a body of a recipient; positioning a cured ring seal adjacent tissue surrounding the opening, wherein the cured ring seal includes an aperture extending there through; inserting an implantable component into the fluidically-sealed body chamber via the opening, wherein the implantable component extends though the aperture in the cured ring seal; and fluidically-sealing the opening around the implantable component with the cured ring seal layer.

In another aspect, an apparatus is provided. The apparatus comprises: a cured polyvinyl alcohol (PVA) ring comprising a body defining a central aperture, wherein the cured PVA ring is configured to be disposed around an implantable component adjacent a surface of an opening in a human body, and wherein the cured PVA ring is configured to expand after implantation to fluidically-seal the space between the implantable component and the surface of the opening.

In another aspect, an apparatus is provided. The apparatus comprises: a cured polyvinyl alcohol (PVA) ring comprising a body defining a central aperture, wherein the cured PVA ring is configured to be disposed around an implantable component adjacent a surface of an opening in a human body, and wherein the cured PVA ring is configured to expand after implantation to fluidically-seal the space between the implantable component and the surface of the opening, wherein the cured PVA ring further comprises at least one longitudinal projection that circumferentially surrounds the aperture and extends in a distal direction, wherein the body of the cured PVA ring further includes a non-perpendicular proximal edge comprising at least one of a chamfered edge, a beveled edge, or a rounded edge, etc. at the transition between a proximal surface of the body and an outer surface of the body, wherein the body of the cured PVA ring further includes a non-perpendicular distal edge comprising at least one of a chamfered edge, a beveled edge, or a rounded edge, etc. at the transition between a distal surface of the body and an outer surface of the body, wherein the body of the cured PVA ring further comprises a distal surface with a curved inner edge, wherein the body of the cured PVA ring is configured to, after implantation and exposure to aqueous media, absorb the aqueous media and swell, wherein the body of the cured PVA ring has a wettability such that absorption of the aqueous media causes the cured PVA ring to develop adhesive properties, and wherein the body of the cured PVA is loaded with one or more therapeutic substances.

### BRIEF DESCRIPTION OF THE DRAWINGS

Embodiments of the present invention are described herein in conjunction with the accompanying drawings, in which:

FIG. 1A is a schematic diagram illustrating the ear of a recipient and a cured ring seal, in accordance with certain embodiments presented herein;

FIG. 1B is a schematic diagram illustrating the cured ring seal with a stimulating assembly inserted into the cochlea shown in FIG. 1A, in accordance with certain embodiments presented herein;

FIG. 2A is a top view of a cured ring seal, in accordance with certain embodiments presented herein;

FIG. 2B is a cross-sectional view of the cured ring seal of FIG. 2A, taken along line 2B-2B;

FIG. 2C is a cross-sectional of the cured ring seal of FIG. 2A in use with a tubular component of an implantable medical device, in accordance with certain embodiments presented herein;

FIG. 3 is a cross-sectional of another cured ring seal in use with a tubular component of an implantable medical device, in accordance with certain embodiments presented herein;

FIG. 4A is a top view of a cured ring seal, in accordance with certain embodiments presented herein;

FIG. 4B is a cross-sectional view of the cured ring seal of FIG. 4A, taken along line 4B-4B;

FIG. 4C is a cross-sectional of the cured ring seal of FIG. 2A in use with a tubular component of an implantable medical device, in accordance with certain embodiments presented herein;

FIG. 5 is a cross-sectional of another cured ring seal in use with a tubular component of an implantable medical device, in accordance with certain embodiments presented herein;

FIG. 6 is a flowchart illustrating an example method, in accordance with certain embodiments presented herein; and

FIG. 7 is a schematic diagram illustrating use of cured ring seal with a vestibular implant, in accordance with certain embodiments presented herein.

### DETAILED DESCRIPTION

The body of an animal, including the body of a human recipient (“recipient”), includes a number of different fluidically-sealed body chambers (e.g., cavities or enclosed areas in which body fluids are sealed). For example, sensitive tissues in the body of a recipient, such as the brain, the ear, the eye, etc. are protected from the normal circulation by fluidic tissue barriers. In particular, the brain is surrounded by the blood-brain barrier (BBB), the inner ear (including the cochlea and the vestibular system) are surrounded by the blood-labyrinth barrier (BLB), and the eye retina is surrounded by the blood-ocular barrier (BOB), which includes the blood-aqueous barrier (BAB) and the blood-retinal barrier (BRB), and so on. Other tissue barriers, such as the round window, and/or the oval window, are also present in the body of a recipient and are two tissue barriers associated with a fluidically-sealed cochlea of a recipient.

An increasing number of implantable medical devices include or comprise components that are permanently or semi-permanently implanted into a number fluidically-sealed body chambers, often through an opening in a tissue barrier (e.g., through the round window, the oval window, a cochleostomy, etc.). Following insertion, the openings through which these implantable components are inserted should be sealed to prevent the fluid within the chamber from leaking out and/or to prevent allow toxins, bacteria, viruses or other components from entering into the chamber, immediately or in the future. As such, presented herein are cured ring seals and related methods for at least temporarily sealing openings in fluidically-sealed body chambers. The cured ring seals include a body that is formed from at least one of a hydrophilic or hygroscopic polymer, such as polyvinyl alcohol (PVA), that is cured prior to implantation into the body of a recipient (e.g., pre-cured). The cured ring seals presented herein are substantially pliable to facilitate positioning around an outer edge/circumference of an implantable component, are self-adhering, and are configured to expand following insertion.

Merely for ease of description, the cured ring seals presented herein will primarily be described with reference to the sealing of a specific fluidically-sealed body chamber of a recipient, namely the cochlea of a recipient behind the round window. However, it is to be appreciated that the cured ring seals presented herein can be used to seal other fluidically-sealed body chambers within the body of a recipient behind other tissue barriers and through different types of openings.

It is also to be appreciated that the cured ring seals and methods presented herein can be used alone or in combination with a number of different types of implantable medical devices. For example, the cured ring seals and methods presented herein may be implemented with auditory prostheses, such as middle ear auditory prostheses, bone conduction devices, direct acoustic stimulators, electro-acoustic prostheses, auditory brain stimulators, cochlear implants, combinations or variations thereof, etc. The cured ring seals and methods presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, cannulas, etc.

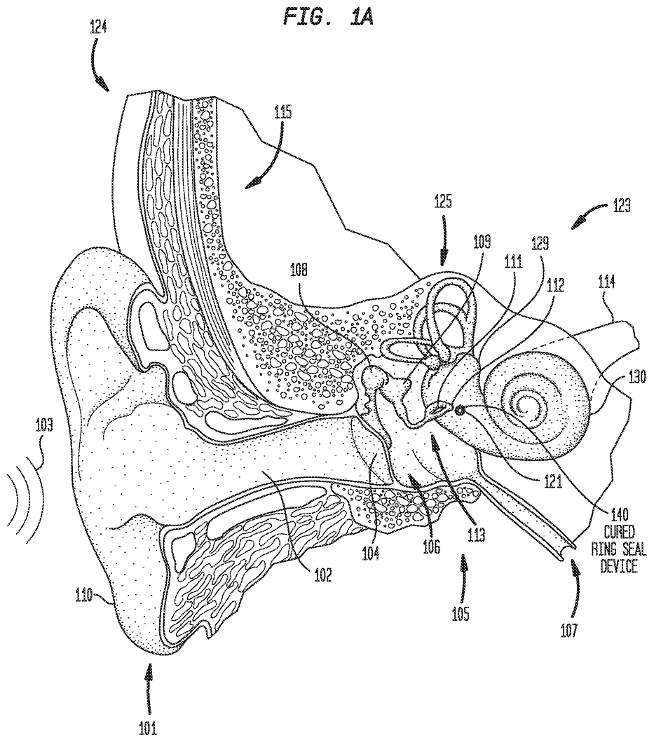

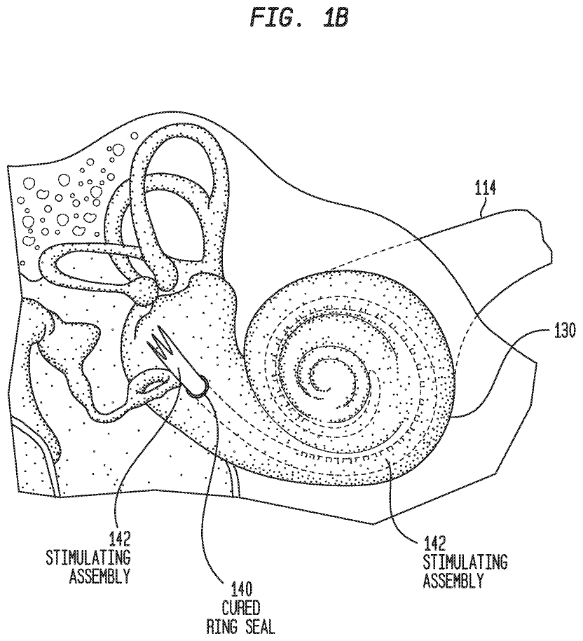

As noted, the cured ring seals are primarily described herein with reference to the sealing of the cochlea of a recipient. Before describing details of the cured ring seals, basic structures of the ear of a recipient, including a cochlea with which a cured ring seal may be used, are first described below with reference to FIGS. 1A and 1B. FIG. 1A illustrates an example cured ring seal configured to seal an opening in the cochlea around an elongate stimulating assembly inserted into the cochlea of the recipient. Merely for ease of illustration, the elongate stimulating assembly has been omitted from FIG. 1A, but the elongate stimulating assembly and cured ring seal are both shown in FIG. 1B.

FIG. 1 illustrates that a recipient's ear normally comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121, through the bones of the middle ear 105. The bones of the middle ear 105 comprise the malleus 108, the incus 109 and the stapes 111, collectively referred to as the ossicles 106. The ossicles 106 are positioned in the middle ear cavity 113 and serve to filter and amplify the sound wave 103, causing oval window 112 to articulate (vibrate) in response to the vibration of tympanic membrane 104. This vibration of the oval window 112 sets up waves of fluid motion of the perilymph within cochlea 130. Such fluid motion, in turn, activates tiny hair cells (not shown) inside of cochlea 130. Activation of the hair cells causes appropriate nerve impulses to be generated and transferred through the spiral ganglion cells (not shown) and auditory nerve 114 to the brain (also not shown) where they are perceived as sound

The human skull is formed from a number of different bones that support various anatomical features. Illustrated in FIG. 1 is the temporal bone 115 which is situated at the side and base of the recipient's skull 124 (covered by a portion of the recipient's skin/muscle/fat, collectively referred to herein as tissue 119). Also shown in FIG. 1 is the bony labyrinth 123, which is a rigid bony structure surrounding the inner ear 107. The bony labyrinth 123 consists of the semicircular canals 125, the vestibule 129, and the cochlea 130, which are chambers/cavities hollowed out of the substance of the bone, and lined by periosteum. The semicircular canals 125, the vestibule 129, and the cochlea 130 each include a corresponding portion of the membranous labyrinth (e.g., the vestibule contains the utricle and saccule, each semicircular canal contains a semicircular duct, and the cochlea contains the cochlear duct). The membranous labyrinth is filled with a fluid called endolymph, and surrounding the membranous labyrinth and filling the remaining space in the bony labyrinth 123 is the perilymph.

The bony labyrinth 123 includes two membrane-covered openings, the oval window 112 (oval window membrane) and the round window 121 (round window membrane). As noted, the oval window 112 vibrates in response to the vibration of tympanic membrane 104. The cochlea 130 is a closed, fluid-filled chamber such that the round window 121 vibrates with opposite phase to the vibrations entering the cochlea 130 through the oval window 112. As such, the round window 121 allows the perilymph in the cochlea 130 to move (in response to vibration at the oval window 112), which in turn ensures that hair cells of the basilar membrane will be stimulated, and that audition will occur. The oval window 112 (oval window membrane) and the round window 121 (round window membrane) are tissue barriers that maintain the fluidic seal of the cochlea 130.

Since the cochlea 130 is a fluidically-sealed body chamber, maintaining the fluidic seal thereof is important to, for example, maintain residual hearing in the cochlea 130, ensure the integrity of the blood-labyrinth barrier, etc. However, as shown in FIG. 1B, cochlear implants utilize a stimulating assembly (e.g., electrode array) 142 inserted into the cochlea 130 via, for example, the oval window 112 or the round window 121 (as shown in FIG. 1), to deliver stimulation signals to nerve cells within the cochlea 130.

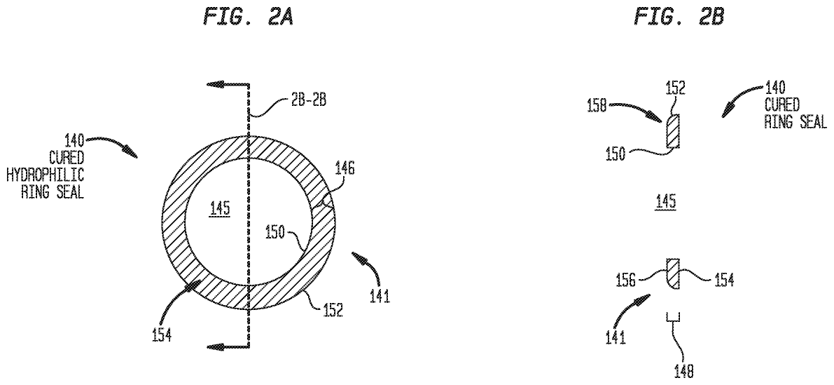

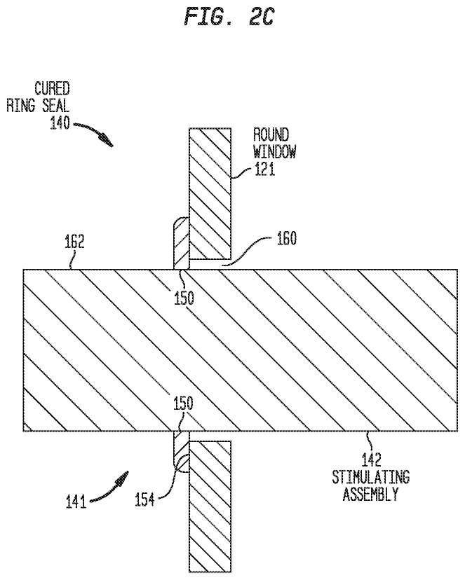

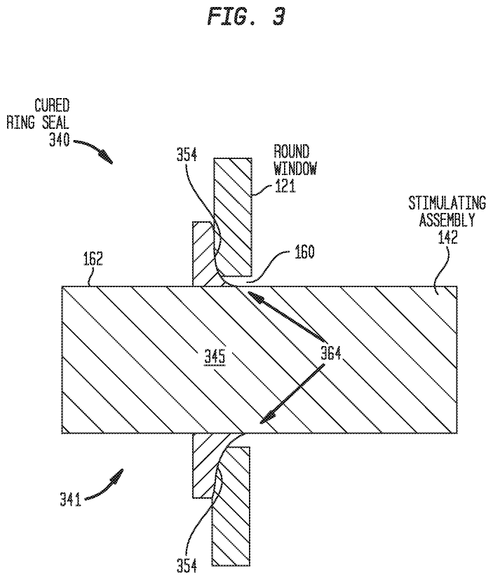

FIG. 1B illustrates that, when implanted in the cochlea 130, the stimulating assembly 142 extends out from the cochlea 132 through the opening via which the stimulating assembly was inserted (e.g., through round window 121). To this end, FIGS. 1A and 1B illustrate the use of a cured (pre-cured) ring seal 140 in accordance with embodiments presented herein. In this example, the cured ring seal 140 is positioned at a proximal surface of the round window 121 and extends around the outer surface (circumference) of the stimulating assembly 140. As described further below, a cured ring seal, such as cured ring seal 140, is configured to at least temporarily fluidically seal the round window 121 (or other opening) around the outer surface of the stimulating assembly 142. Over time (e.g., weeks, months, or years), the cured ring seal 140 can be, for example, resorbed and/or disintegrate and is replaced by a natural seal formed by tissue growth within the body of the recipient. FIGS. 2A, 2B, and 2C are diagrams illustrating further details of the cured ring seal 140.

More specifically, FIG. 2A is a top view of the cured ring seal 140 and the round window 121, while FIG. 2B is cross-sectional view of the cured ring seal 140, taken along line 2B-2B of FIG. 2A. FIG. 2C is a cross-sectional view of the cured ring seal 140 shown in use with a tubular implantable component, namely stimulating assembly 142, at an opening to a fluidically-sealed body chamber, namely at the round window 121.

As shown in FIGS. 2A and 2B, the cured ring seal 140 comprises a body 141 having a general annular-shape and which defines a central aperture 145. The cured ring seal 140 (body 141) has a width 146 and a thickness 148. The width 146 is the dimension of the cured ring seal 140 extending outward from an inner surface 150 of the ring adjacent the central aperture 145 to an outer surface 152 (e.g., the lateral dimension of the cured ring seal 140). The thickness 148 is the dimension of the cured ring seal 140 between a distal surface 154 of the ring seal and a proximal surface 156 of the ring seal (e.g., the longitudinal dimension of the cured ring seal 140). In the examples of FIGS. 2A-2B, the cured ring seal 140 has a thin-film arrangement (e.g., the width 146 is larger than the thickness 148).

In the examples of FIGS. 2A-2C, the outer surface 152 of the cured ring seal 140 includes a non-perpendicular proximal edge 158, meaning that at least a portion of the outer surface 152 adjacent to the proximal surface 156 is not perpendicular to the proximal surface 156. The non-perpendicular proximal edge 158 can be, for example, a chamfered edge, a beveled edge, a rounded edge, etc. at the transition between the proximal surface 156 and the outer surface 152.

The cured ring seal 140 is substantially formed from at least one of a hydrophilic or hygroscopic material, such as a hydrophilic polymer or hygroscopic polymer. In addition, the hydrophilic or hygroscopic material is referred to as being “cured” or “pre-cured,” meaning that the material is “cured” before being implanted into a recipient. As used herein, the “curing” of a hydrophilic or hygroscopic material refers to a phase change in which the hydrophilic or hygroscopic material transitions from a liquid and/or gel form to a substantially solid form. During the curing process of a hydrophilic or hygroscopic material, the material is dried such that water exits the material. In the solid form, referred to herein as the “cured” form, the hydrophilic or hygroscopic material is generally pliable and can be handled by a user (e.g., manually, with instruments/tools, etc.).

Again, as noted, the cured ring seal 140 is substantially formed from at least one of a hydrophilic or hygroscopic material, meaning that the bulk of the material present in the cured ring seal 140 (when implanted, after curing) is the hydrophilic or hygroscopic material. For example, in certain embodiments, at least 75% of the material within the cured ring seal 140 is a hydrophilic or hygroscopic material. In certain embodiments, at least 80% of the material within the cured ring seal 140 is a hydrophilic or hygroscopic material. In certain embodiments, at least 85% of the material within the cured ring seal 140 is a hydrophilic or hygroscopic material. In certain embodiments, at least 90% of the material within the cured ring seal 140 is a hydrophilic or hygroscopic material.

A number of different hydrophilic or hygroscopic materials can be used to form the cured ring seal 140. In one specific example, the cured ring seal 140 is substantially formed from polyvinyl alcohol (PVA). In other examples, the cured ring seal 140 is substantially formed from one or more soluble polymers that are configured to swelling and have adhesive behavior when exposed to aqueous media, such as cellulose ethers, Albumin, Poly(ethylene glycol), Hyaluronic acid, Starch-based biodegradable polymers, Poly (acrylic acid) based polymers, etc.

In certain embodiments, the cured ring seal 140 is loaded/doped with one or more therapeutic substances, such as dexamethasone, CaCO3 to promote bone integration, or another shelf-stable therapeutic substance, that can be released following implantation. The therapeutic substances include, but are not limited, to large and small molecule therapeutic substances, viral vectors or any other type of known therapeutic substances. That is, in the disclosed embodiments, pharmaceutical compositions may comprise any single or combination of the following therapeutic substances: biological substances, bioactive substances, conjugated or fusion molecules or compounds, viral and non-viral vectors, natural, synthetic and recombinant molecules, antibodies and antibody fragments, etc., pharmaceutical agents/active pharmaceutical ingredients (APIs) including commercially available versions of the same, genes, nucleases, endonucleases, nucleic and ribonucleic acids such as messenger RNA (mRNA), siRNA and miRNA, naked DNA, DNA vectors, oligonucleotides, antisense polynucleotides, peptides, polypeptides, proteins including binding proteins, anti-oxidants, and signalling compounds that promote recovery and resolution, other chemicals, ions, and other molecules used to modulate inflammation within the body of individual. A person of ordinary skill in the art will appreciate that each of these substances can be generated by methods known in the art.

Small molecule therapeutic substances include, without limitation, steroids (e.g., dexamethasone, triamcinolone, fluticasone, prednisolone), antibiotics (including aminoglycoside antibiotics, e.g., Kanamycin, Gentamicin), antiapoptotics, antioxidants, antihistamines, anti-inflammatories, NSAID (non-steroidal anti-inflammatories), N-Methyl-D-aspartate (NMDA) receptor antagonists (for treating Tinnitus), therapeutic substance combinations (e.g., FX-322), GSK-3 inhibitors, Wnt activators, sodium thiosulfate (for treating cisplatin-associated ototoxicity, nephrotoxicity and neurotoxicity).

Large molecule therapeutic substances include, without limitation, protein-based therapeutics (therapeutic proteins) including peptides, recombinant proteins, monoclonal antibodies and vaccines, antibody-based therapeutic substances, Fc fusion proteins and other conjugated molecules, anticoagulants, blood factors, bone morphogenetic proteins, engineered protein scaffolds, enzymes, growth factors, hormones including neurotrophins, interferons, interleukins, and thrombolytics.

(API) compounds that are currently in an experimental state include: antioxidants such as HPN-07 and NAC; anesthetics; neurotrophins, mRNA and AAV based gene therapies such as otoferlin and human atonal transcription factors (ATOH1) cDNA, Gamma secreatase inhibitors, JNK stress kinase inhibitors, Kv3 positive modulators; nucleophiles such as sodium thiosulfate pentahydrate to bind to ototoxic compounds such as cisplatin, urea-thiophene carboxamide, 5-HT3 receptor antagonists (e.g., azasetron besylate); ebselen, D-Methionine, Lantanoprost, Xalatan, neurotrophic factors (e.g., BDNF, NT3), Zonasamide and Dendrogenin.

In some embodiments disclosed, one or more therapeutic substantes administered to address the integrity of a tissue barrier, including, among others: (i) vasoconstrictors (e.g., alpha-adrenoceptor agonists, vasopressin analogs, epinephrine, norepinephrine, phenylephrine (Sudafed PE), dopamine, dobutamine, migraine and headache medications (serotonin 5-hydroxytryptamine agonists or triptans)); (ii) corticosteroids (e.g., dexamethasone, bethamethasone, (Celestone), prednisone (Prednisone Intensol), prednisolone (Orapred, Prelone), triamcinolone or triamcinolone-acetonide (Aristospan Intra-Articular, Aristospan Intralesional, Kenalog), and methylprednisolone (Medrol, Depo-Medrol, Solu-Medrol)); (iii) compounds or molecules that have or produce anti-pneumolysin activity to address bacterial-mediated disruption of the brain labyrinth barrier; and (iv) compounds or molecules that have or produce anti caveolin-1 activity, blocking caveolin-1 receptors, or suppressing caveolin-1 over-expression.

Returning to the specific embodiments of FIGS. 2A-2C, as noted above, FIG. 2C illustrates the cured ring seal 140 in use with stimulating assembly 142 at round window 121. As shown, the stimulating assembly 142 extends through an opening 160 in the round window 121 and the cured ring seal 140 is disposed around an outer surface 162 of the stimulating assembly 142. That is, the cured ring seal 140 is shaped such that the stimulating assembly 142 passes through aperture 145 in the cured ring seal 140 and the inner surface 150 of the cured ring seal 140 seats around the periphery of the stimulating assembly. In addition, the distal surface 154 of the cured ring seal 140 is configured to be positioned adjacent (e.g., abutting) the round window 121 (or an adjacent bone). The width 146 of the cured ring seal 140 is sufficiently large to cover a remaining portion of the opening 160 around the stimulating assembly 142.

In accordance with embodiments presented herein, when the cured ring seal 140 is implanted and exposed to aqueous media (e.g., body fluids such as the perilymph or irrigation fluid), the cured ring seal 140 is configured to absorb the aqueous media and swell. The cured ring seal 140 also has a wettability such that absorption of the aqueous media causes the cured ring seal 140 to develop adhesive properties (e.g., a sticky or tacky consistency). In other words, the cured ring seal 140 is configured to be partially hydrolyzed. As noted elsewhere herein, over time (e.g., weeks, months, or years), the cured ring seal 140 can be resorbed, disintegrate, etc. and is replaced by a natural seal formed by tissue growth within the body of the recipient.

The ability of the cured ring seal 140 to develop adhesive properties allows the distal surface 154 to adhere to, and form a fluidic seal with, the proximal surface of the round window 121. In addition, the ability of the cured ring seal 140 to swell causes the ring seal to function as a swelling tourniquet or O-ring around the stimulating assembly 142. That is, the swelling of the cured ring seal 140 causes the ring seal to exert a compressive force on the outer surface 162 of the stimulating assembly 142, thereby forming a fluidic seal between the cured ring seal 140 and the stimulating assembly 142. In addition, the swelling of the cured ring seal 140 causes the ring seal to conform to the shape of the membrane (e.g., to seal any hole, tear or rupture) and can exert a force against the round window 121 to improve the fluidic seal with the round window 121.

As noted, the cured ring seal 140 self-adheres to the round window 121 (or other structure), as well as compresses the stimulating assembly 142. That is, following implantation, the cured ring seal 140 is generally attached to the round window 121 (via the self-adhesion) and attached to the stimulating assembly 142 (via the compression). As such, the cured ring seal 140 can also be configured to function as an anchor to reduce movement of the stimulating assembly. As noted, the cured ring seal 140 can be formulated to elute a therapeutic such as dexamethasone or CaCO3 to promote bone integration or some other shelf stable therapeutic.

In certain examples, the cured ring seal 140 is fully/completely bioresorbable so as to dissolve over time via, for example, hydrolysis. For example, in the case that the cured ring seal 140 is formed from a hydrophilic or hygroscopic polymer, the cured ring seal 140 can be synthetically tailored to include polar functional groups that allow water to break the polymer down to smaller form that accelerates degradation and biological clearance. Poly-lactic-co-glycolic acid, and polyvinyl alcohol are two example, tunable (i.e., rate of degradation can be adjusted), biodegradable polymers. The resorption of the cured ring seal 140 is designed/tuned such that, over time, the cured ring seal 140 is replaced by a natural seal (e.g., via natural tissue growth such as fibrosis) around the opening 160.

An advantage of a fully cured ring seal 140 can be advantageous in that the round window 121 (or other tissue barrier) only needs to be accessed a single time during the initial implantation, and there is no need for a second surgical procedure to remove the cured ring seal 140 after the therapeutic substance(s) have been delivered and/or to leave the surgical site open for an extended period (e.g., to allow time for delivery of the therapeutic substance and subsequent removal).

As noted above, cured ring seals in accordance with embodiments presented herein, such as cured ring seal 140, are configured to be adhered to a tissue barrier and/or bone within the body of a recipient. As such, the cured ring seals needs to be positioned adjacent to the target tissue barrier. A variety of different techniques can be used to position a cured ring seals in accordance with embodiments presented adjacent to the target tissue barrier, including techniques that do or do not make use of surgical devices/instruments.

In certain embodiments, the cured ring seal 140 is positioned adjacent tissue surrounding the round window 121 before implantation of the stimulating assembly 142 and the stimulating assembly is inserted into the opening via the aperture 145 in the cured ring seal 140. In other embodiments, the cured ring seal 140 is positioned around the outer surface 162 of the stimulating assembly 142 before insertion of the stimulating assembly into the cochlea 130 and the stimulating assembly 142 can be inserted into the cochlea 130 until cured ring seal 140 abuts/contacts the tissue surrounding the round window 121. In specific such embodiments, an insertion depth (e.g., angular insertion depth) for the stimulating assembly 142 in the cochlea 130 is determined before insertion and the cured ring seal 140 is positioned around the stimulating assembly 142 at a location on the stimulating assembly that is determined based on the insertion depth for the implantable component. As such, the cured ring seal 140 can operate as a stopper preventing over-insertion of the stimulating assembly 142 into the cochlea 130.

It is to be appreciated that the specific shape and arrangement of the cured ring seal 140 shown in FIGS. 2A-2C is merely illustrative and that the cured ring seals presented herein can have a number of different other shapes and arrangements. For example, FIG. 3 is a cross-sectional view of the cured ring seal 340 shown in use with stimulating assembly 142 at the round window 121. The cured ring seal 340 is similar to the cured ring seal 140 of FIGS. 2A-2C in that it comprises a body 341 having a general annular shape forming a central aperture 345. However, in this example, the cured ring seal 340 (body 341) includes a distal surface 354 with a curved inner edge 364. The curved inner edge 364 of the distal surface 354 forms a lip or projection that circumferentially surrounds the aperture 345 and extends in a distal direction into the opening 160.

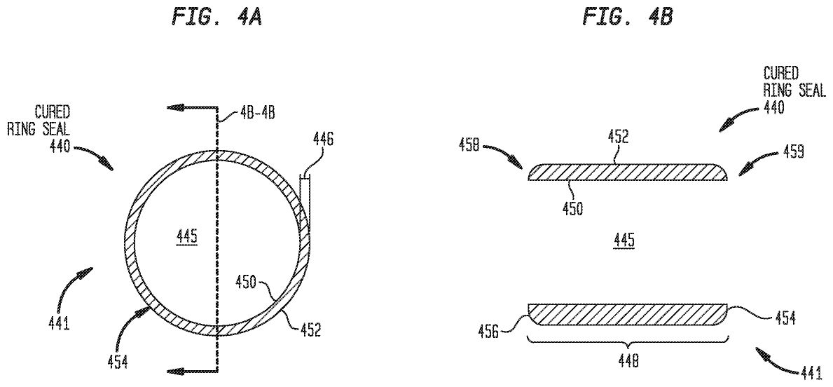

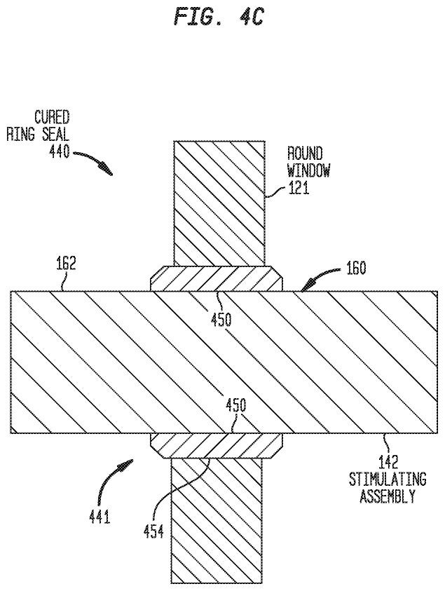

FIGS. 2A-2C and FIG. 3 generally illustrate cured ring seals having a general arrangement in which the ring seal is configured to be placed around an implantable component at the proximal surface of an opening to a fluidically-sealed body chamber. FIGS. 4A-4C illustrate an embodiment of a cured ring seal configured to be placed around an implantable component within an opening to a fluidically-sealed body chamber.

More specifically, FIG. 4A is a top view of the cured ring seal 440 and the round window 121, while FIG. 4B is cross-sectional view of the cured ring seal 440, taken along line 4B-4B of FIG. 4A. FIG. 4C is a cross-sectional view of the cured ring seal 440 shown in use with a tubular implantable component, namely stimulating assembly 142, at an opening to a fluidically-sealed body chamber, namely at the round window 121.

As shown in FIGS. 4A and 4B, the cured ring seal 440 comprises a body 441 having a general annular-shape and defines a central aperture 445. The cured ring seal 440 (body 441) has a width 446 and a thickness 448. The width 446 is the dimension of the cured ring seal 440 extending outward from an inner surface 450 of the ring adjacent the central aperture 445 to an outer surface 452 (e.g., the lateral dimension of the cured ring seal 440). The thickness 448 is the dimension of the cured ring seal 440 between a distal surface 454 of the ring seal and a proximal surface 456 of the ring seal (e.g., the longitudinal dimension of the cured ring seal 440). In the examples of FIGS. 4A-4B, the cured ring seal 440 has a sleeve arrangement where the thickness 448 is larger than the width 446.

In the examples of FIGS. 4A-4C, the outer surface 452 of the cured ring seal 440 includes a non-perpendicular proximal edge 458 and a non-perpendicular distal edge 459. That is, a first portion of the outer surface 452 adjacent to the proximal surface 456 and a second portion of the outer surface 452 adjacent to the distal surface 454 are not perpendicular to the proximal surface 456 and the distal surface 454, respectively. The non-perpendicular proximal edge 458 and a non-perpendicular distal edge 459 can be, for example, chamfered edges, beveled edges, rounded edges, etc. at the transition between the outer surface 452 and each of the proximal surface 456 and the distal surface 454.

Similar to cured ring seal 140, the cured ring seal 440 is also substantially formed from a hydrophilic or hygroscopic material, such as a hydrophilic or hygroscopic polymer, that is “cured,” as described above. Again, as noted, the cured ring seal 440 is substantially formed from a hydrophilic or hygroscopic material, meaning that the bulk of the material present in the cured ring seal 440 (when implanted, after curing) is the hydrophilic or hygroscopic material (e.g., at least 80%, at least 85%, or at least 90% of the material within the cured ring seal 440 is a hydrophilic or hygroscopic material).

A number of different hydrophilic or hygroscopic materials can be used to form the cured ring seal 440. In one specific example, the cured ring seal 440 is substantially formed from polyvinyl alcohol (PVA). In other examples, the cured ring seal 440 is substantially formed from one or more soluble polymers that are configured to swelling and have adhesive behavior when exposed to aqueous media, such as cellulose ethers, Albumin, Poly(ethylene glycol), Hyaluronic acid, Starch-based biodegradable polymers, Poly (acrylic acid) based polymers, etc. Moreover, in certain embodiments, the cured ring seal 440 is loaded/doped with one or more therapeutic substances, such as dexamethasone, CaCO3 to promote bone integration, or another shelf stable therapeutic substance, that can be released following implantation.

Returning to the specific embodiments of FIGS. 4A-4C, as noted above, FIG. 4C illustrates the cured ring seal 440 in use with stimulating assembly 142 at round window 121. As shown, the stimulating assembly 142 extends through an opening 160 in the round window 121 and the cured ring seal 440 is disposed around an outer surface 162 of the stimulating assembly 142. That is, the cured ring seal 440 is shaped such that the stimulating assembly 142 passes through aperture 445 in the cured ring seal 440 and the inner surface 450 of the cured ring seal 440 seats around the periphery of the stimulating assembly.

In addition, in the embodiments of FIGS. 4A-4C, the cured ring seal 440 is configured to be positioned within the opening 160. That is, when implanted, the outer surface 452 of the cured ring seal 440 is configured to be positioned adjacent (e.g., abutting) the round window 121 (or an adjacent bone). The width 446 of the cured ring seal 440 is sufficiently large to substantially fill the portion of the opening 160 around the stimulating assembly 142.

In accordance with embodiments presented herein, when the cured ring seal 440 is implanted and exposed to aqueous media (e.g., body fluids such as the perilymph or irrigation fluid), the cured ring seal 440 is configured to absorb the aqueous media and swell. The cured ring seal 440 also has a wettability such that absorption of the aqueous media causes the cured ring seal 440 to develop adhesive properties (e.g., a sticky or tacky consistency). Over time (e.g., weeks, months, or years), the cured ring seal 440 can be resorbed, disintegrate, etc. and is replaced by a natural seal formed by tissue growth within the body of the recipient.

The ability of the cured ring seal 440 to develop adhesive properties allows the outer surface 452 to adhere to, and form a fluidic seal with, the round window 121. In addition, the ability of the cured ring seal 440 to swell causes the ring seal to function as a swelling tourniquet or O-ring around the stimulating assembly 142. That is, the swelling of the cured ring seal 440 causes the ring seal to exert a compressive force on the outer surface 162 of the stimulating assembly 142, thereby forming a fluidic seal between the cured ring seal 440 and the stimulating assembly 142. In addition, the swelling of the cured ring seal 440 causes the ring seal to conform to the shape of the membrane and can exert an outward force against the round window 121 to improve the fluidic seal with the round window 121.

As noted, the cured ring seal 440 self-adheres to the round window 121 (or other structure), as well as compresses the stimulating assembly 142. That is, the cured ring seal 440 is generally attached to the round window 121 (via the self-adhesion) and attached to the stimulating assembly 142 (via the compression). As such, the cured ring seal 440 can also be configured to function as an anchor to reduce movement of the stimulating assembly. As noted, the cured ring seal 440 can be formulated to elute a therapeutic such as dexamethasone or CaCO3 to promote bone integration or some other shelf stable therapeutic.

In certain examples, the cured ring seal 440 is fully/completely bioresorbable so as to dissolve over time via, for example, hydrolysis. For example, in the case that the cured ring seal 440 is formed from a hydrophilic or hygroscopic polymer, the cured ring seal 440 can be synthetically tailored to include polar functional groups that allow water to break the polymer down to smaller form that accelerates degradation and biological clearance. Poly-lactic-co-glycolic acid, and polyvinyl alcohol are two example, tunable (i.e., rate of degradation can be adjusted), biodegradable polymers. The resorption of the cured ring seal 440 is designed/tuned such that, over time, the cured ring seal 440 is replaced by a natural seal (e.g., via natural tissue growth such as fibrosis) around the opening 160.

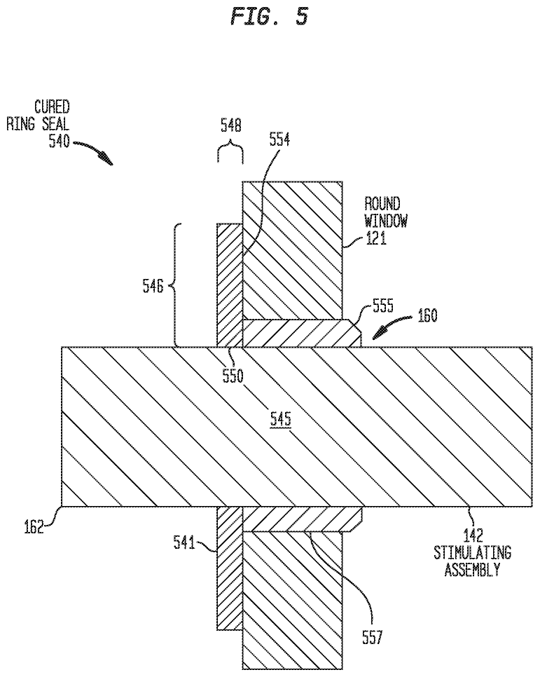

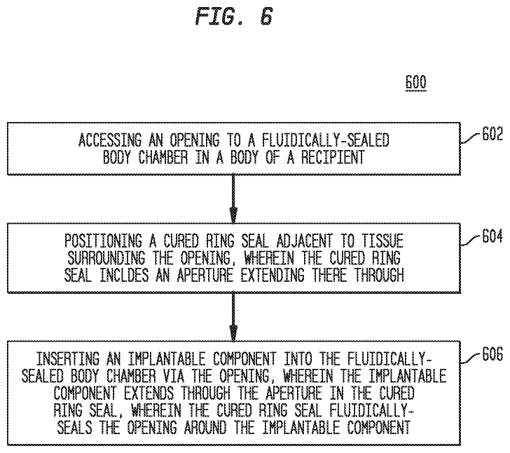

As noted above, FIGS. 2A-2C and FIG. 3 illustrate embodiments of cured ring seals configured to be positioned at a proximal surface of an opening to a fluidically-sealed body chamber. In contrast, FIGS. 4A-4C illustrate embodiments of cured ring seals configured to be positioned within an opening to a fluidically-sealed body chamber. FIG. 5 illustrates an embodiment of a cured ring seal configured to positioned both at a proximal surface of an opening to a fluidically-sealed body chamber and within the opening to the fluidically-sealed body chamber.

More specifically, shown in FIG. 5 is a cured ring seal 540 in use with the stimulating assembly 142 at the round window 121. In this example, the cured ring seal 540 comprises a body 541 that is substantially similar to the cured ring seal 140 of FIGS. 2A-2C. That is, the body 541 has a general annular shape and defines a central aperture 545. The body 541 has a width 546 and a thickness 548.

As shown, the stimulating assembly 142 extends through an opening 160 in the round window 121 and the body 541 is disposed around an outer surface 162 of the stimulating assembly 142. That is, the body 541 is shaped such that the stimulating assembly 142 passes through aperture 545 and the inner surface 550 of the body 541 seats around the periphery of the stimulating assembly. In addition, the distal surface 554 of the cured ring seal 540 is configured to be positioned adjacent the round window 121 (or an adjacent bone).

The cured ring seal 540 also comprises a longitudinal projection 555 that circumferentially surrounds the aperture 545 and extends in a distal direction into the opening 160 from the distal surface 554. That is, the longitudinal projection 555 is configured to be positioned within the opening 160.

In accordance with embodiments presented herein, when the cured ring seal 540 is implanted and exposed to aqueous media (e.g., body fluids such as the perilymph or irrigation fluid), the cured ring seal 540 is configured to absorb the aqueous media and swell. The cured ring seal 540 also has a wettability such that absorption of the aqueous media causes the cured ring seal 540 to develop adhesive properties (e.g., a sticky or tacky consistency).

The ability of the cured ring seal 540 to develop adhesive properties allows the distal surface 554 of the body 541, as well as an outer surface 557 of the longitudinal projection 555, to adhere to, and form a fluidic seal with, the proximal surface of the round window 121. In addition, the ability of the cured ring seal 540 to swell causes the body 541, as well as the longitudinal projection 555, to function as a swelling tourniquet or O-ring around the stimulating assembly 142. That is, the swelling of the cured ring seal 540 causes the ring seal to exert a compressive force on the outer surface 162 of the stimulating assembly 142, thereby forming a fluidic seal between the cured ring seal 540 and the stimulating assembly 142. In addition, the swelling of the cured ring seal 540 causes the ring seal to conform to the shape of the membrane (e.g., to seal any hole, tear or rupture) and can exert a force against the round window 121 to improve the fluidic seal with the round window 121.

FIG. 6 is a flowchart illustrating a method 600, in accordance with certain embodiments presented herein. Method 600 begins at 602 where a user (e.g., surgeon) accesses an opening to a fluidically-sealed body chamber in a body of a recipient. At 604, the user positions a cured ring seal adjacent to tissue surrounding the opening, wherein the cured ring seal includes an aperture extending there through. At 606, the user inserts an implantable component into the fluidically-sealed body chamber via the opening, wherein the implantable component extends though the aperture in the cured ring seal, wherein the cured ring seal fluidically-seals the opening around the implantable component.

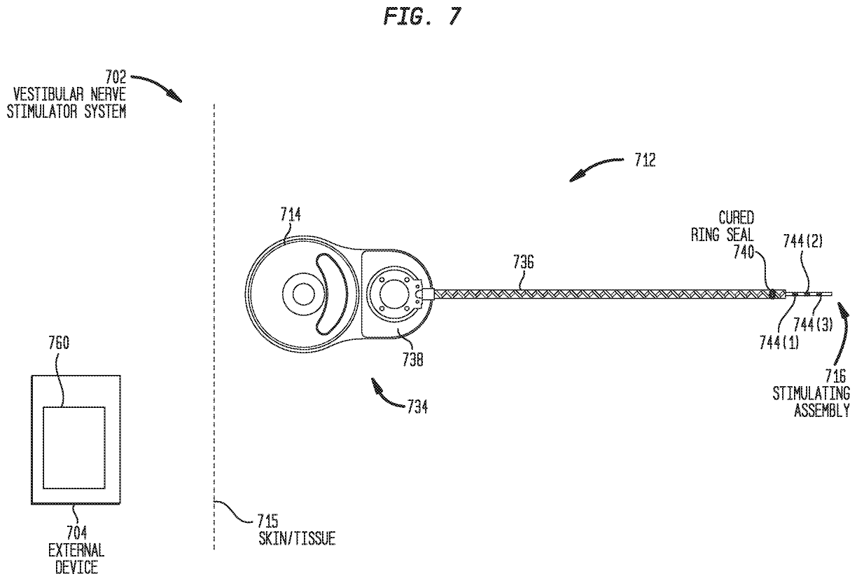

FIG. 7 illustrates an example vestibular stimulator system 702 used with a cured ring seal 740, in accordance embodiments presented herein can be implemented. As shown, the vestibular stimulator system 702 comprises an implantable component (vestibular stimulator) 712 and an external device/component 704 (e.g., external processing device, battery charger, remote control, etc.). The external device 704 comprises a wireless power transmitter unit 760 that may have an arrangement that is similar to, for example, wireless power transmitter units 360 or 860, described above. As such, the external device 704 is configured to transfer power (and potentially data) to the vestibular stimulator 712.

The vestibular stimulator 712 comprises an implant body (main module) 734, a lead region 736, and a stimulating assembly 716, all configured to be implanted under the skin/tissue (tissue) 715 of the recipient. The implant body 734 generally comprises a hermetically-sealed housing 738 in which RF interface circuitry, one or more rechargeable batteries, one or more processors, and a stimulator unit are disposed. The implant body 134 also includes an internal/implantable coil 714 that is generally external to the housing 738, but which is connected to the transceiver via a hermetic feedthrough (not shown).

The stimulating assembly 716 comprises a plurality of electrodes 744 disposed in a carrier member (e.g., a flexible silicone body). In this specific example, the stimulating assembly 716 comprises three (3) stimulation electrodes, referred to as stimulation electrodes 744(1), 744(2), and 744(3). The stimulation electrodes 744(1), 744(2), and 744(3) function as an electrical interface for delivery of electrical stimulation signals to the recipient's vestibular system. It is to be appreciated that this specific embodiment with three stimulation electrodes is merely illustrative and that the techniques presented herein may be used with stimulating assemblies having different numbers of stimulation electrodes, stimulating assemblies having different lengths, etc.

The stimulating assembly 716 is configured such that a surgeon can implant the stimulating assembly within or adjacent to the recipient's vestibular system. For example, the stimulating assembly 716 could be implanted adjacent to the recipient's otolith organs via, for example, the recipient's oval window. In accordance with embodiments presented herein, the cured ring seal 740 is used to seal around the stimulating assembly 716 at the opening (e.g., round window) through which the stimulating assembly 716 is inserted. For ease of illustration, the anatomy of the recipient (e.g., the recipient's vestibular system, round window, etc.) have been omitted from FIG. 7.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1-14. (canceled)

15. A method, comprising:
accessing an opening to a fluidically-sealed body chamber in a body of a recipient;
positioning a cured ring seal adjacent to tissue surrounding the opening, wherein the cured ring seal includes an aperture extending there through; and
inserting an implantable component into the fluidically-sealed body chamber via the opening, wherein the implantable component extends though the aperture in the cured ring seal,
wherein the cured ring seal fluidically-seals the opening around the implantable component.

16. The method of claim 15, further comprising:
positioning the cured ring seal adjacent the tissue surrounding the opening before implantation of the implantable component,
wherein the implantable component is inserted into the opening via the aperture in the cured ring seal.

17. The method of claim 15, further comprising:
positioning the cured ring seal around an outer surface of the implantable component before insertion of the implantable component into the fluidically-sealed body chamber.

18. The method of claim 17, further comprising:
inserting the implantable component into the fluidically-sealed body chamber until the cured ring seal is adjacent the tissue surrounding the opening.

19. The method of claim 18, further comprising:
determining an insertion depth for the implantable component in the fluidically-sealed body chamber; and
positioning the cured ring seal at a location on the implantable component that is determined based on the insertion depth for the implantable component.

20. The method of claim 15, wherein positioning the cured ring seal adjacent tissue surrounding the opening comprises:
positioning the cured ring seal adjacent a proximal surface of the tissue surrounding the opening.

21. The method of claim 15, wherein positioning the cured ring seal adjacent the tissue surrounding the opening comprises:
positioning the cured ring seal within the opening such that that an outer surface of the cured ring seal is adjacent the tissue surrounding the opening.

22. The method of claim 15, further comprising:
self-adhering the cured ring seal to the tissue surrounding the opening.

23. The method of claim 15, further comprising:
delivering one or more therapeutic substances to the fluidically-sealed body chamber via the cured ring seal.

24. (canceled)

25. (canceled)

26. The method of claim 15, wherein fluidically-sealed body chamber is a bone chamber of the recipient.

27. The method of claim 15 wherein fluidically-sealed body chamber is a cochlea of the recipient, and wherein positioning the cured ring seal adjacent to tissue surrounding the opening comprises:
positioning the cured ring seal adjacent at least one of a round window of the cochlea, an oval window of the cochlea, or a surgically-formed cochleostomy.

28. (canceled)

29. (canceled)

30. (canceled)

31. (canceled)

32. An apparatus, comprising:
a cured polyvinyl alcohol (PVA) ring comprising a body defining a central aperture,
wherein the cured PVA ring is configured to be disposed around an implantable component adjacent a surface of an opening in a human body, and wherein the cured PVA ring is configured to expand after implantation to fluidically-seal a space between the implantable component and the surface of the opening.

33. The apparatus of claim 32, wherein the body of the cured PVA ring has a thin-film configuration.

34. (canceled)

35. (canceled)

36. (canceled)

37. (canceled)

38. The apparatus of claim 32, wherein the body of the cured PVA ring is configured to self-adhere to tissue surrounding the opening.

39. (canceled)

40. The apparatus of claim 32, wherein the cured PVA ring further comprises at least one longitudinal projection that circumferentially surrounds the aperture and extends in a distal direction.

41. (canceled)

42. The apparatus of claim 32, wherein the body of the cured PVA ring comprises at least one of a chamfered edge, a beveled edge, or a rounded edge at a transition between a proximal surface of the body and an outer surface of the body, or at least one of a chamfered edge, a beveled edge, or a rounded edge at a transition between a distal surface of the body and an outer surface of the body.

43. (canceled)

44. (canceled)

45. The apparatus of claim 32, wherein the body of the cured PVA ring further comprises a distal surface with a curved inner edge.

46. The apparatus of claim 32, and wherein the body of the cured PVA ring is configured to, after implantation and exposure to aqueous media, absorb the aqueous media and swell.

47. The apparatus of claim 46, wherein the body of the cured PVA ring has a wettability such that absorption of the aqueous media causes the cured PVA ring to develop adhesive properties.

48. The apparatus of claim 32, wherein the body of the cured PVA is loaded with one or more therapeutic substances.

49. (canceled)
